(19) United States
(12) Patent Application Publication
(10) Pub. No.: US 2005/0106729 A1
(43) Pub. Date: May 19, 2005

(54) Gene transfer methods and compositions

(76) Inventors: Mark Irvin Rosenblatt, Cambridge, MA (US); Jin-Hong Chang, Brookline, MA (US); Dimitri T. Azar, Brookline, MA (US)

Correspondence Address: FISH & RICHARDSON PC, 225 FRANKLIN ST, BOSTON, MA, 02110, US

(21) Appl. No.: 10/767,881

(22) Filed: Jan. 29, 2004

Related U.S. Application Data
(63) Continuation of application No. 10/429,549, filed on May 5, 2003, now abandoned.
(60) Provisional application No. 60/377,660, filed on May 3, 2002.

(51) Int. Cl. A61K 48/00; C12N 15/85

(52) U.S. Cl. 435/455; 514/44

(57) ABSTRACT

The invention relates to methods and compositions for transferring genetic material into a cell and regulating expression of the transferred genetic material. Included in the invention are methods for inducing the regulated expression of an exogenous nucleic acid introduced in a cell by the use of a regulator molecule that binds to a protein encoded by a second nucleic acid that has also been introduced into the cell.

### CROSS REFERENCE TO RELATED APPLICATIONS

This application claims priority from U.S. Provisional Patent Application Ser. No. 60/377,660, filed May 3, 2002. The content of the prior application is incorporated herein by reference in its entirety.

### STATEMENT AS TO FEDERALLY SPONSORED RESEARCH

This invention was made with Government support under grant number NEI-10101 awarded by the National Institutes of Health/National Eye Institute. The Government may have certain rights in the invention.

### TECHNICAL FIELD

This invention generally relates to methods and compositions for transferring genetic material into a cell and regulating expression of the transferred genetic material in the cell.

### BACKGROUND OF THE INVENTION

Several strategies have been developed to introduce foreign genes into cells, including direct injection of plasmids or DNA-liposome complexes and infection with modified viruses. However, safety and efficacy are important considerations in the development of therapy protocols that use such gene transfer methods. For example, proteins that are therapeutic in the context of one tissue may be harmful in another. Accordingly, transcriptionally targeted vectors that can restrict the expression of a therapeutic sequence to appropriate cells are particularly desirable. Furthermore, there may in some cases be a therapeutic window for certain proteins, such that levels of expression below or above certain thresholds may be ineffective or toxic, respectively. Therefore, it would also be desirable to create constructs and devise methods that allow exogenous control of expression, so that levels of a therapeutic protein can be raised or lowered according to therapeutic need.

### SUMMARY OF THE INVENTION

The invention is based, at least in part, on the discovery that the expression of an exogenous nucleic acid introduced into a cell can be regulated by the use of a regulator molecule that binds to a protein encoded by a second nucleic acid that has also been introduced into the cell.

In one aspect, the invention features a method of regulating production of a polypeptide in a cell, the method including: introducing into a cell a first nucleotide sequence and a second nucleotide sequence, wherein the first nucleotide sequence (e.g., an expression vector, e.g., a plasmid or viral vector such as a retrovirus) contains a promoter operably linked to a sequence encoding a regulatory element binding protein, wherein the second nucleotide sequence (e.g., an expression vector, e.g., a plasmid or viral vector such as a retrovirus) contains a regulatory element operably linked to a sequence encoding a polypeptide, and wherein the regulatory element binding protein binds to the regulatory element and activates transcription of the sequence encoding the polypeptide upon the binding of a regulator to the regulatory element binding protein; and contacting the cell with an amount of the regulator sufficient to bind to the regulatory element binding protein and activate transcription of the sequence encoding the polypeptide, thereby resulting in transcription of the sequence encoding the polypeptide and production of the polypeptide in the cell.

In another aspect, the invention features, a method of regulating tissue specific production of a polypeptide in a cell, the method including: introducing into a cell a first nucleotide sequence and a second nucleotide sequence, wherein the first nucleotide sequence (e.g., an expression vector, e.g., a plasmid or viral vector such as a retrovirus) contains a promoter operably linked to a sequence encoding a regulatory element binding protein, wherein the second nucleotide sequence (e.g., an expression vector, e.g., a plasmid or viral vector such as a retrovirus) contains a tissue specific promoter and a regulatory element operably linked to a sequence encoding a polypeptide, and wherein the regulatory element binding protein binds to the regulatory element and activates transcription of the sequence encoding the polypeptide upon the binding of a regulator to the regulatory element binding protein; and contacting the cell with an amount of the regulator sufficient to bind to the regulatory element binding protein and activate transcription of the sequence encoding the polypeptide, wherein the cell contains a tissue specific factor that binds to the tissue specific promoter and activates transcription of the sequence encoding the polypeptide, thereby resulting in transcription of the sequence encoding the polypeptide and tissue specific production of the polypeptide in the cell.

In another aspect, the invention features a method of regulating tissue specific production of a polypeptide in a cell, the method including: introducing into a cell a first nucleotide sequence and a second nucleotide sequence, wherein the first nucleotide sequence (e.g., an expression vector, e.g., a plasmid or viral vector such as a retrovirus) contains a tissue specific promoter operably linked to a sequence encoding a regulatory element binding protein, wherein the second nucleotide sequence (e.g., an expression vector, e.g., a plasmid or viral vector such as a retrovirus) contains a regulatory element operably linked to a sequence encoding a polypeptide, and wherein the regulatory element binding protein binds to the regulatory element and activates transcription of the sequence encoding the polypeptide upon the binding of a regulator to the regulatory element binding protein; and contacting the cell with an amount of the regulator sufficient to bind to the regulatory element binding protein and activate transcription of the sequence encoding the polypeptide, wherein the cell contains a tissue specific factor that binds to the tissue specific promoter and activates transcription of the sequence encoding the regulatory element binding protein, thereby resulting in transcription of the sequence encoding the polypeptide and tissue specific production of the polypeptide in the cell.

In another aspect, the invention features a method of inhibiting translation of a target RNA in a cell, the method including: introducing into a cell a first nucleotide sequence and a second nucleotide sequence, wherein the first nucleotide sequence (e.g., an expression vector, e.g., a plasmid or viral vector such as a retrovirus) contains a promoter operably linked to a sequence encoding a regulatory element binding protein, wherein the second nucleotide sequence (e.g., an expression vector, e.g., a plasmid or viral vector such as a retrovirus) contains a regulatory element operably linked to an inhibitory nucleic acid, and wherein the regulatory element binding protein binds to the regulatory element and activates transcription of the inhibitory nucleic acid upon the binding of a regulator to the regulatory element binding protein; and contacting the cell with an amount of the regulator sufficient to bind to the regulatory element binding protein and activate transcription of the inhibitory nucleic acid, thereby resulting in transcription of the inhibitory nucleic acid and hybridization of the inhibitory nucleic acid to a target RNA containing a sequence complementary to the inhibitory nucleic acid, wherein the hybridization inhibits translation of the target RNA.

In another aspect, the invention features a method of inhibiting a biological activity of a target protein in a cell, the method including: introducing into a cell a first nucleotide sequence and a second nucleotide sequence, wherein the first nucleotide sequence (e.g., an expression vector, e.g., a plasmid or viral vector such as a retrovirus) contains a promoter operably linked to a sequence encoding a regulatory element binding protein, wherein the second nucleotide sequence (e.g., an expression vector, e.g., a plasmid or viral vector such as a retrovirus) contains a regulatory element operably linked to a sequence encoding an inhibitory polypeptide, and wherein the regulatory element binding protein binds to the regulatory element and activates transcription of the sequence encoding the inhibitory polypeptide upon the binding of a regulator to the regulatory element binding protein; and contacting the cell with an amount of the regulator sufficient to bind to the regulatory element binding protein and activate transcription of the sequence encoding the inhibitory polypeptide, thereby resulting in transcription of the sequence encoding the inhibitory polypeptide, production of the inhibitory polypeptide in the cell, and binding of the inhibitory polypeptide to a target protein to thereby inhibit a biological activity of the target protein in the cell.

In another aspect, the invention features a composition (e.g., a kit) containing any two of the nucleic acids described herein (e.g., any of the first nucleotide sequences and second nucleotide sequences described herein). Also included is a composition containing at least one or two nucleic acids described herein and a regulator described herein. A composition can also include instructions for the use of the nucleic acids and/or regulator for inducing regulated gene expression.

In another aspect, the invention features a cell (e.g., an isolated cell) containing a first expression vector and a second expression vector, wherein the first expression vector contains a promoter operably linked to a sequence encoding a regulatory element binding protein, wherein the second expression vector contains a regulatory element operably linked to a sequence encoding a polypeptide, and wherein the regulatory element binding protein binds to the regulatory element and activates transcription of the sequence encoding the polypeptide upon the binding of a regulator to the regulatory element binding protein.

The invention also includes a method of regulating production of a polypeptide in the cell by contacting the cell with an amount of the regulator sufficient to bind to the regulatory element binding protein and activate transcription of the sequence encoding the polypeptide, thereby resulting in transcription of the sequence encoding the polypeptide and production of the polypeptide in the cell.

In another aspect, the invention features a cell (e.g., an isolated cell) containing a first expression vector and a second expression vector, wherein the first expression vector contains a promoter operably linked to a sequence encoding a regulatory element binding protein, wherein the second expression vector contains a tissue specific promoter and a regulatory element operably linked to a sequence encoding a polypeptide, wherein the regulatory element binding protein binds to the regulatory element and activates transcription of the sequence encoding the polypeptide upon the binding of a regulator to the regulatory element binding protein, and wherein the cell contains a tissue specific factor that binds to the tissue specific promoter and activates transcription of the sequence encoding the polypeptide.

The invention also includes a method of regulating tissue specific production of a polypeptide in the cell by contacting the cell with an amount of the regulator sufficient to bind to the regulatory element binding protein and activate transcription of the sequence encoding the polypeptide, thereby resulting in transcription of the sequence encoding the polypeptide and tissue specific production of the polypeptide in the cell.

In another aspect, the invention features a cell (e.g., an isolated cell) containing a first expression vector and a second expression vector, wherein the first expression vector contains a tissue specific promoter operably linked to a sequence encoding a regulatory element binding protein, wherein the second expression vector contains a regulatory element operably linked to a sequence encoding a polypeptide, wherein the regulatory element binding protein binds to the regulatory element and activates transcription of the sequence encoding the polypeptide upon the binding of a regulator to the regulatory element binding protein, and wherein the cell contains a tissue specific factor that binds to the tissue specific promoter and activates transcription of the sequence encoding the regulatory element binding protein.

The invention also includes a method of regulating tissue specific production of a polypeptide in the cell by contacting the cell with an amount of the regulator sufficient to bind to the regulatory element binding protein and activate transcription of the sequence encoding the polypeptide, thereby resulting in transcription of the sequence encoding the polypeptide and tissue specific production of the polypeptide in the cell.

In another aspect, the invention features a cell (e.g., an isolated cell) containing a first expression vector and a second expression vector, wherein the first expression vector contains a promoter operably linked to a sequence encoding a regulatory element binding protein, wherein the second expression vector contains a regulatory element operably linked to an inhibitory nucleic acid, and wherein the regulatory element binding protein binds to the regulatory element and activates transcription of the inhibitory nucleic acid upon the binding of a regulator to the regulatory element binding protein.

The invention also includes a method of inhibiting translation of a target RNA in the cell by contacting the cell with an amount of the regulator sufficient to bind to the regulatory element binding protein and activate transcription of the inhibitory nucleic acid, thereby resulting in transcription of the inhibitory nucleic acid and hybridization of the inhibitory nucleic acid to a target RNA containing a sequence complementary to the inhibitory nucleic acid, wherein the hybridization inhibits translation of the target RNA.

In another aspect, the invention features a cell (e.g., an isolated cell) containing a first expression vector and a second expression vector, wherein the first expression vector contains a promoter operably linked to a sequence encoding a regulatory element binding protein, wherein the second expression vector contains a regulatory element operably linked to a sequence encoding an inhibitory polypeptide; and wherein the regulatory element binding protein binds to the regulatory element and activates transcription of the sequence encoding the inhibitory polypeptide upon the binding of a regulator to the regulatory element binding protein.

The invention also includes a method of inhibiting a biological activity of a target protein in the cell by contacting the cell with an amount of the regulator sufficient to bind to the regulatory element binding protein and activate transcription of the sequence encoding the inhibitory polypeptide, thereby resulting in transcription of the sequence encoding the inhibitory polypeptide, production of the inhibitory polypeptide in the cell, and binding of the inhibitory polypeptide to a target protein to thereby inhibit a biological activity of the target protein in the cell.

In another aspect, the invention features a transgenic, non-human mammal (e.g., a mouse, rat, or goat) whose genome comprises: (1) any first nucleotide sequence (e.g., expression vector) described herein; (2) any second nucleotide sequence (e.g., expression vector) described herein; or (3) any combination of first and second nucleotide sequences (e.g., expression vectors) described herein.

“Operably linked” refers to any linkage (regardless of orientation or distance) between an expression control sequence and coding (or non-coding) sequence, where the linkage permits the expression control sequence to control transcription of the coding (or non-coding) sequence.

A “regulatory element binding protein” is a protein that, upon binding to a regulator, binds to a regulatory element and activates transcription of a sequence operably linked to the regulatory element. An example of a regulatory element binding protein is a steroid hormone receptor, e.g., an ecdysone hormone receptor.

A “regulatory element” is a sequence of nucleotides operably linked to a coding (or non-coding) sequence that, when bound by a regulatory element binding protein, activates transcription of the linked coding (or non-coding) sequence. An example of a regulatory element is a steroid hormone receptor-regulated promoter, e.g., an ecdysone hormone receptor promoter.

A “regulator” is a molecule that binds to regulatory element binding protein, thereby causing the regulatory element binding protein to bind to a regulatory element. A regulator can be any molecule that binds to the regulatory element binding protein, e.g., a polypeptide, a small peptide, or a small molecule. An example of a regulator is a steroid hormone, e.g., ecdysone, or a steroid analog.

The compositions and methods descried herein allow for regulated gene transfer to corneal cells, ocular surfaces, and other tissues. The methods descried herein can avoid either inadequate expression of a gene or overexpression of a gene, either of which may be undesirable during the treatment of disease or during the regulation of biological processes. For example, uncontrolled expression could cause unwanted, dangerous side effects. The systems described herein provide an advance in that they allow for careful regulation of biological process or disease processes not afforded by unregulated expression. For example, controlled expression of an exogenous nucleic acid in the cornea could be achieved by topical application of a safe drug without the need for repeated gene transfer treatments.

The invention provides a means to control the expression of genes for the treatment of a variety of diseases. The methods may be especially useful in those diseases which require the capacity for long-term gene expression, but which also require careful control of expression to provide ideal therapeutic effect without unwarranted side effects from gene overexpression.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, the preferred methods and materials are described below. All publications, patent applications, patents, and other references mentioned herein are incorporated by reference in their entirety. In case of conflict, the present application, including definitions, will control. The materials, methods, and examples are illustrative only and not intended to be limiting.

Other features and advantages of the invention will be apparent from the following detailed description, and from the claims.

### BRIEF DESCRIPTION OF THE DRAWINGS

FIG. 1 depicts nucleic acid constructs usable in a non-tissue specific regulatable system.

FIG. 2 depicts nucleic acid constructs usable in a tissue specific regulatable system containing a tissue specific promoter with an intervening regulating element.

FIG. 3 depicts nucleic acid constructs usable in a tissue specific regulatable system containing a tissue specific promoter driving expression of a regulatory element binding protein.

FIG. 4 depicts nucleic acid constructs usable in a system for regulation of inhibitory RNA expression.

FIG. 5 depicts nucleic acid constructs usable in a system for regulation of inhibitory gene products.

### DETAILED DESCRIPTION

The present invention provides methods and compositions for obtaining regulated expression of an exogenous nucleic acid in a cell. Such controlled expression methods are useful in that they allow for the regulated expression of a nucleic acid encoding a polypeptide, e.g., a therapeutic polypeptide, in an amount and/or for a duration desired to achieve a desired biological result, e.g., a therapeutic effect. In addition, regulated gene expression is of wide general interest to biologists as such a system can be used to permit the evaluation of biological effects of a protein product by turning on or off expression of a nucleic acid encoding the polypeptide.

As described in the accompanying Example, a system of gene transfer to corneal and ocular surface tissues has been developed that allows for regulatable expression of exogenous genes. The transferred nucleic acids include elements of a regulatable system, wherein the gene to be regulated is placed downstream of a regulatable promoter (e.g., an ecdysone or tetracycline response element). The gene required for regulation of the regulatable promoter (e.g., an ecdysone hormone receptor or a tetracycline binding protein) can be constitutively expressed in the system using standard high expression promoters (e.g., a cytomegalovirus promoter). Following transfer of a given gene into a cell, expression can be regulated in a dose dependent manner by application of a regulating substance (e.g., ecdysone, tetracycline, or their respective analogs) to the cell, e.g., the corneal surface or to culture medium, with responses occurring in a dose dependent manner. These systems can allow for tightly controlled regulation of genes in tissues such as, for example, the cornea, other ocular tissues, the ocular surface, skin, and other tissues or organs.

The following are non-limiting examples of designs of expression systems that can be used for the nucleic acids constructs described herein. The components of the following systems can be interchanged and used in a variety of combinations.

Non-Tissue Specific Regulatable System

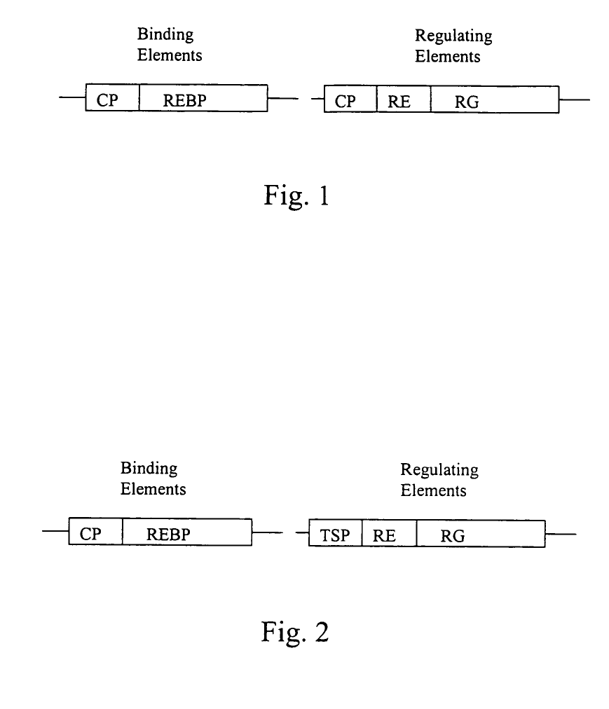

FIG. 1 depicts an example of a non-tissue specific regulatable expression system. In this example, the following abbreviations are used: CP—Constitutive Promoter; REBP—Gene for Regulatory Element Binding Protein; RE—Regulatory Element; and RG—Regulated Gene.

Expression of the Regulated Gene can be regulated in a cell containing the two nucleic acid constructs depicted in FIG. 1 by regulating the exposure of the cell to a Regulator. The Regulator is molecule that binds to the Regulatory Element Binding Protein, thereby causing the Regulatory Element Binding Protein to bind to the Regulatory Element and activate expression of the Regulated Gene. Accordingly, the Regulated Gene is not actively transcribed when the cell is not exposed to the Regulator, whereas transcription of the Regulated Gene is activated when the cell is exposed to the Regulator.

In the system of FIG. 1, the two nucleic acid constructs can be used to achieve regulated gene expression in many tissue types due to the use of a promoter that is constitutively active. Accordingly, the Constitutive Promoter can be any promoter sequence that is known to induce expression of a gene sequence in a wide variety of tissues. For example, a β-actin promoter or a cytomegalovirus promoter can be used in such a construct. The Constitutive Promoter can be operably linked to the Gene for Regulatory Element Binding Protein and/or to the Regulated Gene. In some cases, the Regulatory Element is the only sequence used to control expression of the Regulated Gene.

Tissue Specific Regulatable System

A regulatable system in conjunction with a tissue-specific promoter can be used to achieve regulated gene expression in a tissue-specific manner. Such tissue specific methods can, at least partially, avoid the untoward effects of expression of genes in tissues surrounding those that are to be targeted.

A large number of tissue specific promoters have been identified. Examples of such promoters include, but are not limited to, the keratocan promoter which is expressed in keratocytes, the TIE1 promoter which is expressed in vascular endotheliurn, and promoters involved in transporters in the corneal endothelium.

FIG. 2 depicts an example of a system using a tissue specific promoter with an intervening regulating element. In this example, the following abbreviations are used: CP—Constitutive Promoter; TSP—Tissue Specific Promoter; REBP—Gene for Regulatory Element Binding Protein; RE—Regulatory Element; and RG—Regulated Gene.

Expression of the Regulated Gene can be regulated in a cell containing the two nucleic acid constructs depicted in FIG. 2 by regulating the exposure of the cell to a Regulator in a manner similar to that described herein with respect to FIG. 1. However, in this example, in addition to the presence or absence of the Regulator, the cell type that contains the two nucleic acid constructs also determines whether the Regulated Gene will be expressed. For example, if the Tissue Specific Promoter is active in cell type A but not in cell type B, then the introduction of the two nucleic acid constructs depicted in FIG. 2 into both cell types A and B, followed by the contacting of cell types A and B with the Regulator, will result in activated expression of the Regulated Gene only in cell type A.

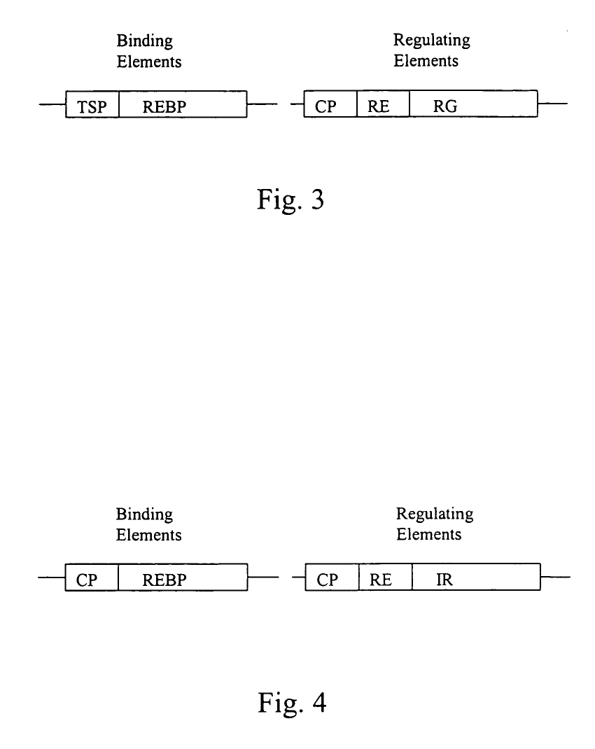

FIG. 3 depicts an example of a system using a tissue specific promoter driving the expression of the Regulating Element Binding Protein. In this example, the following abbreviations are used: CP—Constitutive Promoter; TSP—Tissue Specific Promoter; REBP—Gene for Regulatory Element Binding Protein; RE—Regulatory Element; and RG—Regulated Gene.

The system depicted in FIG. 3, like that of FIG. 2, allows for the regulated expression of the Regulated Gene by means of the exposure to the Regulator in a tissue specific manner. In FIG. 3, expression of the Regulating Element Binding Protein is dependent upon the tissue type into which the nucleic acid constructs are introduced.

Regulatable Gene “Knockout”

A regulatable system described herein can also be used to downregulate or “knockout” a protein expressed by a cell, tissue, or organism (e.g., a non-human mammal such as a mouse, rat, or goat). The regulation methods of the invention can reduce or eliminate possible side effects associated with complete “knockout” of gene products that are required in at least minimal quantities for physiologic function.

FIG. 4 depicts the regulation of inhibitory RNA expression. In this example, the following abbreviations are used: CP—Constitutive Promoter; REBP—Gene for Regulatory Element Binding Protein; RE—Regulatory Element; and IR—Inhibitory RNA.

Expression of the Inhibitory RNA can be regulated in a cell containing the two nucleic acid constructs depicted in FIG. 4 by regulating the exposure of the cell to a Regulator in a manner similar to that described herein with respect to FIG. 1. In this system, the Inhibitory RNA can bind to a specific nucleic acid, e.g., a nucleic acid encoding a protein of interest, thereby inhibiting translation or transcription of the sequence. For example, regulated expression of an Inhibitory RNA that binds to an expressed mRNA can prevent translation or accelerate degradation of the mRNA. The methods include, for example, expression of an antisense strand of a cDNA or the production of small iRNA.

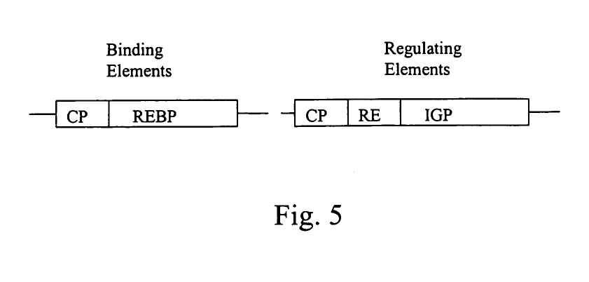

FIG. 5 depicts the regulated expression of inhibitory gene products. In this example, the following abbreviations are used: CP—Constitutive Promoter; REBP—Gene for Regulatory Element Binding Protein; RE—Regulatory Element; and IGP—Inhibitory Gene Product.

Expression of the Inhibitory Gene Product can be regulated in a cell containing the two nucleic acid constructs depicted in FIG. 5 by regulating the exposure of the cell to a Regulator in a manner similar to that described herein with respect to FIG. 1. In this system, the Inhibitory Gene Product can cause the inhibition of a biological activity of a protein of interest. For example, the Inhibitory Gene Product can bind to and inactivate a protein of interest. Included in the method is the regulated expression of a nucleotide sequence encoding a dominant negative and/or inhibitory protein. Such methods can cause the functional “knockout” of a protein in a controlled fashion. Controlled knockouts can be particularly useful when the knockout of a gene in an entire animal results in a non-viable animal.

Methods of Gene Transfer and Routes of Administration for Nucleic Acids and Regulators

The invention encompasses systems and methods for the in vitro, in vivo and/or ex vivo delivery of nucleic acids to a cell, e.g., a corneal cell or a cell at an ocular surface, accompanied by the regulated expression of the nucleic acid in the cell. A variety of methods for introducing nucleic acids into a cell are described herein are well know to those of skill in the art.

A nucleic acid can be naked or associated or complexed with a delivery vehicle. For a description of the use of naked DNA, see e.g., U.S. Pat. No. 5,693,622. For example, naked DNA constructs of the sort described herein can be injected at high pressure into a tissue such as corneal stroma.

Nucleic acids can be delivered to a cell using delivery vehicles known in the art, such as lipids, depot systems, hydrogel networks, particulates, liposomes, ISCOMS, microspheres or nanospheres, microcapsules, microparticles, gold particles, virus-like particles, nanoparticles, polymers, condensing agents, polysaccharides, polyamino acids, dendrimers, saponins, adsorption enhancing materials, colloidal suspensions, dispersions, powders, or fatty acids.

Viral particles can also be used, e.g., retroviruses, adenovirus, adeno-associated virus, pox viruses, SV40 virus, alpha virus, lentivirus, or herpes viruses.

For in vivo administration, the dosage for any given patient depends upon many factors, including the patient's size, body surface area, age, the particular nucleic acid to be administered, sex, time and route of administration, general health, and other drugs being administered concurrently. Determination of optimal dosage is well within the abilities of a pharmacologist of ordinary skill.

Other standard delivery methods, e.g., biolistic transfer, or ex vivo treatment, can also be used. In ex vivo treatment, cells can be obtained from a patient or an appropriate donor and treated ex vivo with nucleic acid constructs described herein, and then returned to the patient.

Microparticles or nanoparticles can be used as vehicles for delivering nucleic acids into cells. The microparticles can contain macromolecules embedded in a polymeric matrix or enclosed in a shell of polymer. Microparticles act to maintain the integrity of the macromolecule, e.g., by maintaining the DNA in a nondegraded state.

The nucleic acids can be administered into subjects via lipids, dendrimers, microspheres, colloids, suspensions, emulsions, depot systems, hydrogel networks, liposomes, or electroporation using techniques that are well known in the art. For example, liposomes carrying nucleic acids can be delivered as described in Reddy et al. (1992) J. Immunol. 148:1585; Collins et al. (1992) J. Immunol. 148:3336-3341; Fries et al. (1992) Proc. Natl. Acad. Sci. USA 89:358; and Nabel et al. (1992) Proc. Nat. Acad. Sci. USA 89:5157.

The nucleic acid constructs described herein can be administered by using ISCOMS, which are negatively charged, cage-like structures of 30-40 nm in size formed spontaneously on mixing cholesterol and Quil A (saponin), or saponin alone. The nucleic acids can also be electroporated into cells or tissues of a recipient. Electroporation may occur ex vivo or in vivo.

For in vivo administration, nucleic acids and/or Regulators can be administered to a subject in any manner known in the art, e.g., intramuscularly, intravenously, intraarterially, intradermally, intraperitoneally, intranasally, intravaginally, intrarectally or subcutaneously, or they can be introduced into the gastrointestinal tract, the mucosa, or the respiratory tract, e.g., by inhalation of a solution or powder containing the nucleic acids and/or Regulators.

Nucleic acids and/or Regulators can be applied topically (e.g., to the ocular surface, skin, joints, or gastrointestinal system), into the anterior chamber, into the vitreous, transclerally, or systemically (oral or intravenous). Nucleic acids and/or Regulators can be delivered in a pharmaceutically acceptable carrier such as saline. Nucleic acids and Regulators can either be administered simultaneously or at separate times. For example, a nucleic acid can first be administered to an individual, followed by administrations of the Regulator at specific periods when expression of the nucleic acid is desired.

The use of this regulated gene system is applicable to regulated expression of genes in vitro and in vivo in ocular tissues as well as other tissues and organs. Examples include, but are not limited to, the following:
(1) Regulated expression of genes in conjunctival cells, e.g., to restore/augment tear components in dry eye syndromes;
(2) Regulated expression of antiangiogenic proteins in corneal epithelium and/or keratocytes, e.g., to inhibit neovascularization following injury, and during ocular inflammation;
(3) Regulated expression of genes allowing proliferation of endothelial cells in Fuchs' Dystrophy;
(4) Regulated gene expression in the ciliary body/iris, e.g., to allow for regulation of genes to control intraocular pressure;
(5) Regulated gene expression of antiangiogenic or antiproliferative compounds in the retina, e.g., to control proliferative vitreoretinopathy and age-related macular degeneration;
(6) Regulated expression of genes delivered to the optic nerve head, e.g., to provide neuroprotection against the progression of glaucomatous optic nerve damage;
(7) Regulated expression of genes delivered to the skin or gastrointestinal tract, e.g., to treat skin or gastrointestinal disease; and
(8) Regulated gene expression of genes delivered to the joints, e.g., to control recurrent and episodic arthritis.

Regulation gene expression in vitro in these tissues/organs, or cells derived from these tissue or organs, can also be useful in the development of therapeutic or diagnostic agents.

Table 1 lists examples of routes of gene application and routes of regulator application that can be used for a variety of tissues and disease conditions associated with the listed tissues. Similar applications may be designed for other tissues and organs.

TABLE 1
Methods of Gene Application and Regulator Administration

| | Route of Gene | Route of Regulator |
|---|---|---|
| Tissue Type | Application | Application |
| Corneal | Injection into limbus | Topical eye drops or |
| epithelium/ | | subconjunctival injection |
| limbal cells | | |
| Conjunctiva | Subconjunctival injection | Topical eye drops or |
| | | subconjunctival injection |
| Keratocytes | Intrastromal injection | Topical eye drops or |
| | | subconjunctival injection |
| Corneal | Anterior chamber | Topical eye drops or |
| endothelium | injection | subconjunctival injection |
| Ciliary Body | Anterior chamber | Topical eye drops or |
| | injection | subconjunctival injection |
| Retina | Intravitreal or subretinal | Sub-Tenon's injection |
| | injection; transcleral | |
| Skin | Intradermal injection | Topical |
| Gastrointestinal | Submucosal injection | Oral |
| Joints | Intra-articular injection | Topical |

This invention is further illustrated by the following example that should not be construed as limiting.

### EXAMPLE

Regulation of MMP-7 Expression in MMP-7 Deficient Corneal Epithelial Cells Using an Ecdysone Regulated Ecotropic Retroviral Vector System

The present example demonstrates stable gene transfer and regulated gene expression in a corneal epithelial cell line using ecotropic retroviral vectors.

A cDNA encoding mouse pro matrix metalloproteinase-7 (MMP-7) was obtained from the I.M.A.G.E. clone consortium. Using a PCR-based cloning strategy, the cDNA was inserted into the multiple cloning site of pCFP-EGSH (a plasmid vector including retroviral packaging signals and a multiple cloning site downstream of an ecdysone hormone receptor responsive promoter) to make plasmid pCFB-EGSH-MMP-7. A second plasmid used in these experiments was pFB-ERV, a plasmid vector with retroviral packaging signals, and expressing the constituents of functional ecdysone hormone receptors capable of binding ponasterone A and activating the ecdysone responsive promoter in pCFP-EGSH.

Replication deficient retroviral supematants from pCGB-EGSH-MMP-7 and pFB-EGSH were produced separately. The plasmid for pCFB-EGSH-MMP-7 or pFB-EGSH was co-transfected using a liposomal method into HEK293 cells along with the plasmids pEnv (expressing a retroviral ecotropic envelope protein) and pGag-Pol (expressing the gag and pol genes required for assembly of infectious retroviral particles). Supernatants from these cells contained replication deficient retrovirus capable of transferring genes into suitable murine target cells.

Retroviral supernatants containing CFB-EGSH-MMP-7 and FB-ERV replication deficient retroviruses mixed in a 1:1 molar ratio were used to infect subconfluent MMP-7 deficient corneal epithelial cells (immortalized cells from an MMP-7 knockout mouse) in vitro.

MMP-7 deficient corneal epithelial cells co-infected with CFB-EGSH-MMP-7 and FB-ERV replication deficient retroviruses were induced with 10 μM ponasterone A. an ecdysone analog and a ligand for the ecdysone receptor. Immunohistochemistry was performed on induced and non-induced two days later using a primary rabbit polyclonal antibody against MMP-7 and a secondary FITC conjugated antibody. Immunolocalization was determined using epifluorescence microscopy. Expression of MMP-7 was seen only in infected epithelial cells induced with ponasterone A and, not in non-induced infected epithelial cells.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of regulating production of a polypeptide in a cell, the method comprising:
introducing into a cell a first nucleotide sequence and a second nucleotide sequence, wherein the first nucleotide sequence comprises a promoter operably linked to a sequence encoding a regulatory element binding protein, wherein the second nucleotide sequence comprises a regulatory element operably linked to a sequence encoding a polypeptide, and wherein the regulatory element binding protein binds to the regulatory element and activates transcription of the sequence encoding the polypeptide upon the binding of a regulator to the regulatory element binding protein; and
contacting the cell with an amount of the regulator sufficient to bind to the regulatory element binding protein and activate transcription of the sequence encoding the polypeptide,
thereby resulting in transcription of the sequence encoding the polypeptide and production of the polypeptide in the cell.

2. A method of regulating tissue specific production of a polypeptide in a cell, the method comprising:
introducing into a cell a first nucleotide sequence and a second nucleotide sequence, wherein the first nucleotide sequence comprises a promoter operably linked to a sequence encoding a regulatory element binding protein, wherein the second nucleotide sequence comprises a tissue specific promoter and a regulatory element operably linked to a sequence encoding a polypeptide, and wherein the regulatory element binding protein binds to the regulatory element and activates transcription of the sequence encoding the polypeptide upon the binding of a regulator to the regulatory element binding protein; and
contacting the cell with an amount of the regulator sufficient to bind to the regulatory element binding protein and activate transcription of the sequence encoding the polypeptide, wherein the cell contains a tissue specific factor that binds to the tissue specific promoter and activates transcription of the sequence encoding the polypeptide,
thereby resulting in transcription of the sequence encoding the polypeptide and tissue specific production of the polypeptide in the cell.

3. A method of regulating tissue specific production of a polypeptide in a cell, the method comprising:
introducing into a cell a first nucleotide sequence and a second nucleotide sequence, wherein the first nucleotide sequence comprises a tissue specific promoter operably linked to a sequence encoding a regulatory element binding protein, wherein the second nucleotide sequence comprises a regulatory element operably linked to a sequence encoding a polypeptide, and wherein the regulatory element binding protein binds to the regulatory element and activates transcription of the sequence encoding the polypeptide upon the binding of a regulator to the regulatory element binding protein; and
contacting the cell with an amount of the regulator sufficient to bind to the regulatory element binding protein and activate transcription of the sequence encoding the polypeptide, wherein the cell contains a tissue specific factor that binds to the tissue specific promoter and activates transcription of the sequence encoding the regulatory element binding protein,
thereby resulting in transcription of the sequence encoding the polypeptide and tissue specific production of the polypeptide in the cell.

4. A method of inhibiting translation of a target RNA in a cell, the method comprising:
introducing into a cell a first nucleotide sequence and a second nucleotide sequence, wherein the first nucleotide sequence comprises a promoter operably linked to a sequence encoding a regulatory element binding protein, wherein the second nucleotide sequence comprises a regulatory element operably linked to an inhibitory nucleic acid, and wherein the regulatory element binding protein binds to the regulatory element and activates transcription of the inhibitory nucleic acid upon the binding of a regulator to the regulatory element binding protein; and
contacting the cell with an amount of the regulator sufficient to bind to the regulatory element binding protein and activate transcription of the inhibitory nucleic acid,
thereby resulting in transcription of the inhibitory nucleic acid and hybridization of the inhibitory nucleic acid to a target RNA comprising a sequence complementary to the inhibitory nucleic acid, wherein the hybridization inhibits translation of the target RNA.

5. A method of inhibiting a biological activity of a target protein in a cell, the method comprising:
introducing into a cell a first nucleotide sequence and a second nucleotide sequence, wherein the first nucleotide sequence comprises a promoter operably linked to a sequence encoding a regulatory element binding protein, wherein the second nucleotide sequence comprises a regulatory element operably linked to a sequence encoding an inhibitory polypeptide, and wherein the regulatory element binding protein binds to the regulatory element and activates transcription of the sequence encoding the inhibitory polypeptide upon the binding of a regulator to the regulatory element binding protein; and
contacting the cell with an amount of the regulator sufficient to bind to the regulatory element binding protein and activate transcription of the sequence encoding the inhibitory polypeptide,
thereby resulting in transcription of the sequence encoding the inhibitory polypeptide, production of the inhibitory polypeptide in the cell, and binding of the inhibitory polypeptide to a target protein to thereby inhibit a biological activity of the target protein in the cell.

6. A cell comprising a first expression vector and a second expression vector, wherein the first expression vector comprises a promoter operably linked to a sequence encoding a regulatory element binding protein, wherein the second expression vector comprises a regulatory element operably linked to a sequence encoding a polypeptide, and wherein the regulatory element binding protein binds to the regulatory element and activates transcription of the sequence encoding the polypeptide upon the binding of a regulator to the regulatory element binding protein.

7. A cell comprising a first expression vector and a second expression vector, wherein the first expression vector comprises a promoter operably linked to a sequence encoding a regulatory element binding protein, wherein the second expression vector comprises a tissue specific promoter and a regulatory element operably linked to a sequence encoding a polypeptide, wherein the regulatory element binding protein binds to the regulatory element and activates transcription of the sequence encoding the polypeptide upon the binding of a regulator to the regulatory element binding protein, and wherein the cell contains a tissue specific factor that binds to the tissue specific promoter and activates transcription of the sequence encoding the polypeptide.

8. A cell comprising a first expression vector and a second expression vector, wherein the first expression vector comprises a tissue specific promoter operably linked to a sequence encoding a regulatory element binding protein, wherein the second expression vector comprises a regulatory element operably linked to a sequence encoding a polypeptide, wherein the regulatory element binding protein binds to the regulatory element and activates transcription of the sequence encoding the polypeptide upon the binding of a regulator to the regulatory element binding protein, and wherein the cell contains a tissue specific factor that binds to the tissue specific promoter and activates transcription of the sequence encoding the regulatory element binding protein.

9. A cell comprising a first expression vector and a second expression vector, wherein the first expression vector comprises a promoter operably linked to a sequence encoding a regulatory element binding protein, wherein the second expression vector comprises a regulatory element operably linked to an inhibitory nucleic acid, and wherein the regulatory element binding protein binds to the regulatory element and activates transcription of the inhibitory nucleic acid upon the binding of a regulator to the regulatory element binding protein.

10. A cell comprising a first expression vector and a second expression vector, wherein the first expression vector comprises a promoter operably linked to a sequence encoding a regulatory element binding protein, wherein the second expression vector comprises a regulatory element operably linked to a sequence encoding an inhibitory polypeptide, and wherein the regulatory element binding protein binds to the regulatory element and activates transcription of the sequence encoding the inhibitory polypeptide upon the binding of a regulator to the regulatory element binding protein.

11. A method of regulating production of a polypeptide in a cell, the method comprising contacting the cell of claim 6 with an amount of the regulator sufficient to bind to the regulatory element binding protein and activate transcription of the sequence encoding the polypeptide, thereby resulting in transcription of the sequence encoding the polpeptide and production of the polypeptide in the cell.

12. A method of regulating tissue specific production of a polypeptide in a cell, the method comprising contacting the cell of claim 7 with an amount of the regulator sufficient to bind to the regulatory element binding protein and activate transcription of the sequence encoding the polypeptide, thereby resulting in transcription of the sequence encoding the polypeptide and tissue specific production of the polypeptide in the cell.

13. A method of regulating tissue specific production of a polypeptide in a cell, the method comprising contacting the cell of claim 8 with an amount of the regulator sufficient to bind to the regulatory element binding protein and activate transcription of the sequence encoding the polypeptide, thereby resulting in transcription of the sequence encoding the polypeptide and tissue specific production of the polypeptide in the cell.

14. A method of inhibiting translation of a target RNA in a cell, the method comprising contacting the cell of claim 9 with an amount of the regulator sufficient to bind to the regulatory element binding protein and activate transcription of the inhibitory nucleic acid, thereby resulting in transcription of the inhibitory nucleic acid and hybridization of the inhibitory nucleic acid to a target RNA comprising a sequence complementary to the inhibitory nucleic acid, wherein the hybridization inhibits translation of the target RNA.

15. A method of inhibiting a biological activity of a target protein in a cell, the method comprising contacting the cell of claim 10 with an amount of the regulator sufficient to bind to the regulatory element binding protein and activate transcription of the sequence encoding the inhibitory polypeptide, thereby resulting in transcription of the sequence encoding the inhibitory polypeptide, production of the inhibitory polypeptide in the cell, and binding of the inhibitory polypeptide to a target protein to thereby inhibit a biological activity of the target protein in the cell.
